(19) United States
(12) Patent Application Publication
(10) Pub. No.: US 2021/0275983 A1
(43) Pub. Date: Sep. 9, 2021

(54) DIELECTRIC PACKING MATERIAL FOR CONVERSION OF CARBON DIOXIDE TO VALUABLE MATERIALS BY NON-THERMAL PLASMA TECHNOLOGY

(71) Applicant: Kyu Bum Han, Seoul (KR)

(72) Inventor: Kyu Bum Han, Seoul (KR)

(21) Appl. No.: 17/190,473

(22) Filed: Mar. 3, 2021

Related U.S. Application Data
(60) Provisional application No. 62/984,368, filed on Mar. 3, 2020.

(51) Int. Cl. B01J 15/00; B01J 23/52; C07C 27/06; B01J 23/62; B01J 23/60; B01J 23/02

(52) U.S. Cl. CPC B01J 15/005; B01J 23/52; B01J 23/02; B01J 23/62; B01J 23/60; C07C 27/06

(57) ABSTRACT

The present invention relates to a dielectric packing material for converting carbon dioxide to a valuable material using non-thermal plasma technology, and more particularly, to a dielectric packing material for converting carbon dioxide to a valuable material using non-thermal plasma technology, wherein the dielectric packing material is packed in a non-thermal plasma reactor for conversion of carbon dioxide to a valuable material and is formed to have a hollow structure with multiple edges on the surface thereof to effectively scatter non-thermal plasma at the edges and thereby to improve CO₂ conversion and energy efficiency.

### CROSS-REFERENCE TO RELATED APPLICATION

The benefit under 35 USC § 119 of U.S. Provisional Patent Application 62/984,368 of Kyu Bum HAN filed Mar. 3, 2020 for “DIELECTRIC PACKING MATERIAL FOR CONVERSION OF CARBON DIOXIDE TO VALUABLE MATERIALS BY NON-THERMAL PLASMA TECHNOLOGY” is hereby claimed. The disclosure of U.S. Provisional Patent Application 62/984,368 is hereby incorporated herein by reference, in its entirety, for all purposes.

### TECHNICAL FIELD

The present invention relates to a dielectric packing material for converting carbon dioxide to a valuable material using non-thermal plasma technology. More particularly, the present invention relates to a dielectric packing material for converting carbon dioxide into a valuable material using non-thermal plasma technology, wherein the dielectric packing material is packed in a non-thermal plasma reactor for conversion of carbon dioxide to valuable substances and is formed to have a hollow structure with multiple edges on the surface thereof to effectively scatter non-thermal plasma through the edges and thereby to improve CO₂ conversion and energy efficiency.

### BACKGROUND ART

The development of modern society has brought about a continual increase in the use of fossil fuels. As a result, there is increasing global interest in the global environment consequences of discharging a huge amount of gas. In particular, carbon dioxide (CO₂) emitted from sources such as transportation petroleum, coal/fossil fuel combustion, and natural processes, is considered to be a major greenhouse gas causing global warming. The U.S. Energy Information Administration reported that over 5 billion metric tons of CO₂ was emitted in the United States in 2018. Efforts to reduce carbon dioxide emissions continue through international agreements. In addition, with the adoption of the Kyoto Protocol, which regulates the emission of greenhouse gases such as CO₂, moratoria on greenhouse gas (GHG) emissions and regulations associated with violation of GHG emissions have begun in earnest. Since the degree to which CO₂, among greenhouse gases, contributes to the greenhouse effect is about 50%, regulating CO₂ emission means controlling the greenhouse effect.

Various methods for treating CO₂ are being examined. Interest in chemically recycling CO₂, as one method, is gradually increasing. Chemically recycling CO₂ refers to a method that includes separating and recovering emitted CO₂ and converting the CO₂ into other valuable compounds for the production of fuels and fine chemicals and the synthesis of polymers using a catalyst and the like. In particular, many studies are underway on the synthesis of methanol and valuable substances having two or more carbon atoms through hydrogenation of CO₂.

Converting CO₂ into methanol for use as a fuel, fuel additive or precursor is an effective alternative sink for CO₂ (Kumaran Gnanamani, M. et al., Green Carbon Dioxide 99-118 (2014)). This conversion has the potential to be used in the recycling of commodity chemicals and fuels and atmospheric CO₂ (Alvarado, M. IHS Chem. Bull. 3, 10-11 (2016)). Several alternative CO₂-to-methanol conversion technologies including plasma, electrochemical, solar thermochemical, photochemical, and biochemical conversion have been investigated (Snoeckx, R. & Bogaerts, A. Chem. Soc. Rev. 46, 5805-5863 (2017)). Current industrial processes employ high pressures (30 atm-300 atm) and high temperatures (200° C.-300° C.) (Jadhav, S. G. et al., Chem. Eng. Res. Des. 92, 2557-2567 (2014)). The main advantage of plasma technology over traditional CO₂ conversion pathways is that it is a low-temperature process. Low-temperature plasma or non-thermal plasma is created by applying a potential difference across two electrodes connected in parallel in a reactor filled with gas. The difference causes breakdown of gas, creating free radicals through dissociation induced by collisions between electrons and ions (Alvarado, M. IHS Chem. Bull. 3, 10-11 (2016). This reaction proceeds with low energy consumption and high CO₂ conversion efficiency.

A non-thermal plasma reactor is an attractive system for direct conversion of CO₂ into valuable fuel. For example, the non-thermal plasma reactor can be operated at ambient temperature owing to the highly energetic electrons. The flexible process allows the plasma reactor to be instantaneously switched on and off with conversion and a short stabilization time for product yield. Due to low investment cost and independence from rare earth materials, the non-thermal plasma reactor is easily scaled up from watt to megawatt applications. While many types of plasma reactor are available, the dielectric barrier discharge (DBD) plasma reactor has a high potential for use in economically viable processes. The reactor has a simple design is easy to use and promotes sustainability in a single channel. The DBD reactor shows a wide range of total conversion or the sum of the effective conversions. However, the energy cost for CO₂ conversion is below the efficiency target for conversion into syngas. When suitable catalysts are applied to the reactor, the energy costs will be shifted down to the target because a DBD reactor requires one-step process and allows the direct production of valuable oxygenates at high yield. The direct hydrogenation of CO₂ with H₂ is a significant route for CO₂ conversion due to reduced thermodynamic limitations compared to direct CO₂ decomposition and dry reforming of methane (DRM). Furthermore, the direct production of methanol from CO₂ has advantages such as a reduced amount of byproducts and lower energy requirements in product purification (Marlin, D. S. et al., Front. Chem. 6, 446 (2018)).

Meanwhile, there remains a trade-off between conversion and energy efficiencies in non-thermal plasma-based CO₂ processes. The non-thermal plasma reactor is packed with a dielectric packing material (DPM) in the presence or absence of a catalyst. A number of studies have reported that CO₂ conversion with additional inert gases dilutes pure CO₂ with the assistance of DBD plasma (Indarto, A. et al., J. Hazard. Mater. 146, 309-315 (2007); Okazaki, K. Catal. Today 211, 29-38 (2013)). This process produces unwanted byproducts, which is not preferable in industrial applications (Ozkan, A. et al. J. CO2 Util. 9, 74-81 (2015)). To prevent the generation of such byproducts, other studies have focused on conversion without dilution by adding different dielectric materials such as aluminum oxide (Al₂O₃), zirconium dioxide (ZrO₂), and calcium titanate (CaTiO₃); the CO₂ conversion improved the energy efficiency of the plasma process (Tu, X. & Whitehead, J. C., Appl. Catal. B Environ. 125, 439-448 (2012)). Furthermore, the DBD with dielectric packing materials can be operated at ambient pressure and low temperature. This advantage of dielectric packing materials increases the potential for industrial applications. The presence of a packing material enhances the energy efficiency of the reactor and reduces the breakdown voltage required for plasma ignition by strengthening the electric field near the contact points in the discharge region. The average power density of a packed reactor is higher than that of an unpacked reactor, thereby yielding a high decomposition of gas chemicals. The packing material shape influences the capacitance of the plasma reactor and changing the discharge (Chang, J. S. et al., Annual Report Conference on Electrical Insulation and Dielectric Phenomena (Cat. No. 98CH36257) 2, 485-488 vol. 2 (1998)).

Studies of plasma technology at low temperature have tried to rectify this by adding a catalyst, but the technology still has a trade-off between conversion rate and energy efficiency. The catalyst shape is adhered to spherical dielectric materials. The geometry of dielectric materials directly influences energy efficiency, and the spherical shape is not the optimal geometrical shape for energy efficiency in a DBD plasma system (Veerapandian, S. et al., Catalysts 7, 113 (2017); Ray, D. & Subrahmanyam, C. RSC Adv. 6, 39492-39499 (2016)). In addition, disadvantageously, catalysts are inactivated due to phase change upon high-temperature reaction, and problems such as sintering of metal components and deposition of carbon over time as well as resistance against coke formation.

Accordingly, as a result of extensive efforts to solve the above problems, the present inventors found that, when a dielectric packing material packed in a non-thermal plasma reactor for converting carbon dioxide to methanol is formed to have a hollow structure with multiple edges on the surface thereof, non-thermal plasma can be effectively scattered by the edges, and CO₂ conversion and energy efficiency can be improved. Based on this finding, the present invention has been completed.

### SUMMARY OF THE INVENTION

It is an object of the present invention to provide a dielectric packing material for converting carbon dioxide (CO₂) into a valuable material including methanol that is capable of remarkably improving CO₂ conversion and energy efficiency even under the conditions of low temperature (<100° C.) and atmospheric pressure, a catalyst composite including the same, and a method for producing the same.

It is another object of the present invention to provide a device for producing valuable materials from carbon dioxide using the dielectric packing material, a catalyst composite containing the same, and a method of producing valuable materials from carbon dioxide using the same.

In accordance with one aspect of the present invention, the above and other objects can be accomplished by the provision of a dielectric packing material for converting carbon dioxide to valuable materials by scattering non-thermal plasma, having a hollow cylindrical structure and multiple grooves on a surface thereof.

In accordance with another aspect of the present invention, there is provided a catalyst composite for converting carbon dioxide to valuable materials by scattering non-thermal plasma, the catalyst composite having a structure in which a surface of the dielectric packing material described above is coated with a catalyst.

In accordance with another aspect of the present invention, there is provided a method of producing the dielectric packing material for converting carbon dioxide to valuable materials by scattering non-thermal plasma, the method comprising performing a reduction reaction by sintering a dielectric packing material at a temperature of 1,000° C. to 1,200° C. and a partial pressure of oxygen (P_{O2}) of 10⁻⁸ to 10⁻⁶ atm.

In accordance with another aspect of the present invention, there is provided a method of preparing the catalyst composite described above, the method including coating the dielectric packing material described above with a catalyst.

In accordance with yet another aspect of the present invention, there is provided a device for preparing valuable materials from carbon dioxide using a dielectric barrier discharge plasma system, the device including a reactor body, an internal electrode and an external electrode provided in the reactor, an inlet tube for supplying reactants into the reactor, a power supply for supplying current to the internal electrode and the external electrode to generate plasma, and a ground portion of the current connected to the external electrode, wherein the reactor body is packed with the dielectric packing material described above or the catalyst composite described above.

In accordance with yet another aspect of the present invention, there is provided a method of preparing valuable materials from carbon dioxide, the method including (a) packing a reactor body with the dielectric packing material described above or the catalyst composite described above and supplying a carbon-dioxide-containing gas mixture thereto, (b) heating the supplied gas mixture; and (c) preparing valuable materials by applying a high voltage to an internal electrode and an external electrode provided in the reactor.

### BRIEF DESCRIPTION OF THE DRAWINGS

The above and other objects, features and other advantages of the present invention will be more clearly understood from the following detailed description taken in conjunction with the accompanying drawings, in which:

FIG. 1A is a side view (a) and a top view (b) of a dielectric packing material according to an embodiment of the present invention;

FIG. 1B is a side view (a) and a top view (b) of a dielectric packing material coated with a catalyst according to another embodiment of the present invention (blue: coated catalyst);

FIG. 2 shows a potential reaction scheme for producing methanol through a plasma-catalyst CO₂ hydrogenation process;

FIG. 3 is a schematic diagram illustrating a plasma device for converting CO₂ into methanol and an analytic process using gas chromatography (GC) and mass spectrometry (MS) according to an embodiment of the present invention; and

FIG. 4 is a cross-sectional image showing Al₂O₃ ceramic beads added with iron oxide in the dielectric packing material according to an embodiment of the present invention, wherein the bead was sintered at (a) 1250° C./1.8×10⁻¹³ atm P_{O2} and (b) 1450° C./1.8×10⁻¹¹ P_{O2}.

### DESCRIPTION OF ABBREVIATION

DPM: Dielectric Packing Materials with or without catalyst
MFC: Mass Flow Controller
MFM: Mass Flowmeter
CM: Current Monitor
EC: External Capacitor
GC: Gas Chromatography
MS: Mass Spectroscopy

### DETAILED DESCRIPTION OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

The present invention is based on the finding that, when a dielectric packing material packed in a non-thermal plasma reactor for converting carbon dioxide to methanol is formed to have a hollow structure with multiple edges on the surface thereof, non-thermal plasma can be effectively scattered by the edges, and CO₂ conversion and energy efficiency can be improved.

Accordingly, in one aspect, the present invention is directed to a dielectric packing material for converting carbon dioxide to methanol through non-thermal plasma technology, the dielectric packing material having a hollow cylindrical structure having multiple edges on the surface thereof.

In another aspect, the present invention is directed to a method of producing a dielectric packing material for converting carbon dioxide to methanol through non-thermal plasma technology, the method including sintering a dielectric packing material at a temperature of 1,000° C. to 1,200° C. and an oxygen partial pressure (P_{O2}) of 10⁻⁸ atm to 10⁻⁶ atm to perform a reduction reaction.

Hereinafter, the present invention will be described in detail.

The present invention is capable of improving energy efficiency and CO₂ conversion rate by optimizing the shape of the dielectric packing material. The dielectric packing material according to the present invention has a hollow cylindrical shape with multiple sharp edges. The dielectric packing material optimizes the electrical field with regard to plasma and gas flow rate, which will increase the energy efficiency and CO₂ conversion.

In the present invention, the dielectric packing material may include at least one selected from the group consisting of Al₂O₃ including γ-Al₂O₃, ZrO₂, CaO, MgO, zeolite (HZSM-5, ZSM-5, or the like), quartz sand, and glass beads.

In the present invention, the dielectric packing material may have a particle size of 100 μm to 500 μm.

The plasma reactor is packed with a dielectric packing material, with or without a catalyst. The present invention is characterized by the structure/shape of the dielectric packing material for optimizing the energy efficiency and carbon dioxide conversion rate. The dielectric packing material, which is coated or not coated with a catalyst, plays an important role in improving the DBD reactor's energy efficiency. Many dielectric packing materials were tested in the course of the present research, among which ZrO₂ and CaO exhibited the highest conversion (30-45%) and energy efficiency (5-10%). Moreover, a particle size of 180 μm to 300 μm can increase CO₂ conversion by up to 70% (Snoeckx, R. & Bogaerts, A., Chem. Soc. Rev. 46, 5805-5863 (2017)). As the particle size is reduced, the reactor has a higher chance of occurrence of breakdown voltage and partial discharge. Variation in the shape of the dielectric packing material (DPM) may influence the capacitance of the plasma reactor, which changes the discharge characteristics and energy efficiency. For example, the efficiency is increased by changing the shape from a sphere to a hollow cylinder. The gas flow pressure drop of the packed-bed reactor is reduced by changing the shape of DPM from a sphere to a hollow cylinder. As a result, the peak current of the microdischarge with the hollow cylinder DPM is greater than that of the spherical shape ((Chang, J. S. et al., Annual Report Conference on Electrical Insulation and Dielectric Phenomena (Cat. No. 98CH36257) 2, 485-488 vol. 2 (1998)). The enhancement of the electrical field is higher for a hollow cylinder than for a spherical product due to the presence of a sharp edge (Veerapandian, S. et al., Catalysts 7, 113 (2017); Takaki, K. et al., IEEE Trans. Plasma Sci. 32, 2175-2183 (2004)).

As described above, according to the present invention, multiple sharp edges may be created on a hollow cylindrical DPM surface so that the energy efficiency of a plasma reactor is optimized by enhancing the electrical field. The optimized efficiency directly influences conversion efficiency.

In another aspect, the present invention is directed to a catalyst composite for converting carbon dioxide to valuable materials through non-thermal plasma technology, the catalyst composite having a structure in which a surface of the dielectric packing material described above is coated with a catalyst.

In another aspect, the present invention is directed to a method of preparing a catalyst composite for converting carbon dioxide to valuable materials through non-thermal plasma technology, the method including coating the dielectric packing material described above with a catalyst.

In the present invention, the catalyst may be selected from the group consisting of Cu, Pt, Pd, Au, In₂O₃, ZnO, BaTiO₃ and/or TiO₂. Preferably, the catalyst is Cu, Au, Pd—ZnO, Pt—In₂O₃, BaTiO₃, Cu—In₂O₃/TiO₂, Cu—ZnO or the like.

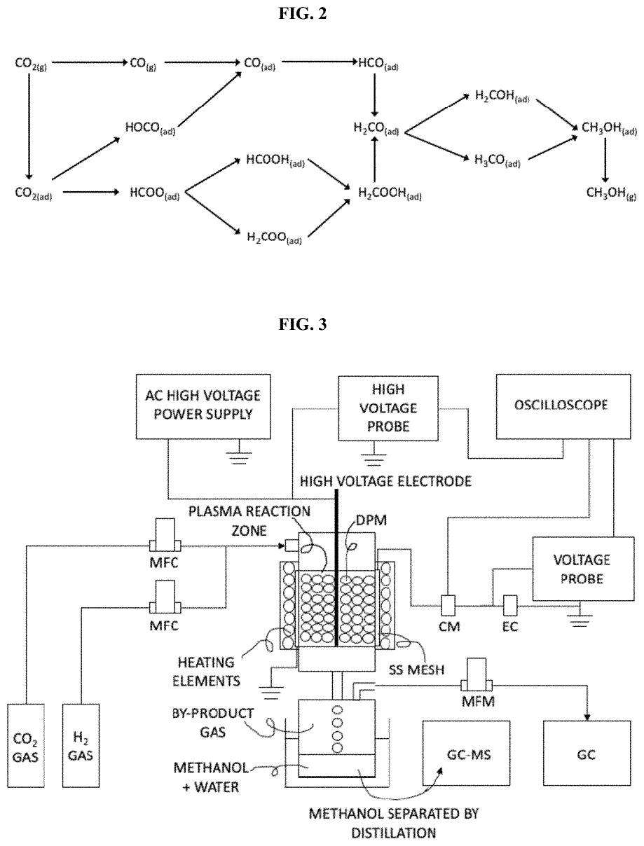

It is a challenge to simultaneously realize all of energy efficiency, CO₂ conversion efficiency, and selectivity. A catalyst package consisting of the dielectric packing material (DPM) and a chemical catalyst may be produced. The catalyst is capable of increasing selectivity, but the catalyst material in a nano-powder form cannot alone catalyze the reaction due to the weak binding of CO₂ to the catalyst (Kattel, S. et al., J. Catal. 343, 115-126 (2016)). It is important to add a catalyst to a dielectric packing material for conversion efficiency and selectivity. A possible reaction pathway on a catalyst surface for hydrogenation of CO₂ to methanol is shown in FIG. 2. The use of a catalyst can drastically alter the chemical pathways compared to an empty plasma reaction.

At elevated temperature and pressure, methanol selectivity up to 50% is easily achieved. However, at 30° C. and atmospheric pressure, most catalysts including ordinary catalysts have difficulty reaching 50% selectivity. Copper (Cu) is still observed to be the most promising catalyst material for methanol selectivity. Copper (Cu), platinum (Pt), and indium oxide (In₂O₃) were selected as catalysts for comparison.

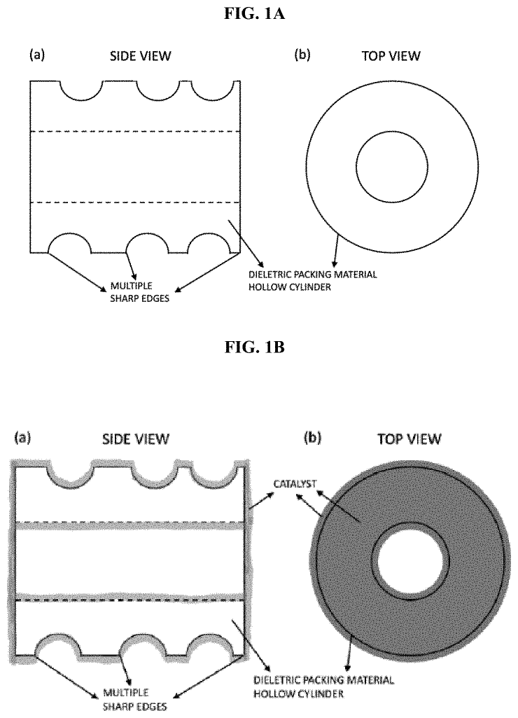

For selectivity, the DPM surface is coated with diverse chemical catalysts, and the most promising catalyst is selected. The expected catalyst package is shown in FIG. 1. As can be seen from the side view of FIG. 1A, multiple sharp edges, created by a reduction reaction under a low partial pressure of oxygen, are formed on the DPM surface. To further facilitate the reaction, the selected catalyst (blue in FIG. 1) is coated on the DPM surface and hollow.

To create multiple sharp edges on the dielectric packing material, a reduction reaction is conducted at a low partial pressure of oxygen (P_{O2}). Technology for forming micron pores in iron oxide added aluminum oxide (Al₂O₃) ceramic beads is developed. The pore size and porosity are controllable by balancing temperature and P_{O2}. A higher sintering temperature with a lower P_{O2}, for example, generates a larger pore size and higher porosity (FIG. 4). This technology is applied to the surface of DPM. By maximally increasing the electrical field, the multiple sharp edges are expected to increase energy efficiency and CO₂ conversion efficiency. For chemical catalyst adherence, dip coating may be conducted on the DPM. This technique is easily scalable from laboratory scale to pilot due to the simplicity of the process and the relatively short processing time.

The diameter, length, and size of each geometry are measured under optimized conditions. Methanol conversion was used to identify the optimized geometry and shape. Furthermore, the porosity may affect methanol conversion. For instance, the packing material using a DBD system highly enhanced the electrical field in the hollow cylinder due to the sharp edge. Al₂O₃ reduction may create pores in the ceramic material (Kim, H.-H., Plasma Process. Polym. 1, 91-110 (2004); Bogaerts, A. & C. Neyts, E., ACS Energy Lett. 3, 1013-1027 (2018)). Al₂O₃ reduction may create pores in ceramic materials. For example, according to an Ellingham diagram, pores are formed in an iron-oxide-embedded alumina micro-bead at 1450° C. and at 10⁻⁷¹ P_{O2} atm, and the porosity can be controlled by varying the temperature and partial pressure of oxygen (FIG. 4). According to an Ellingham diagram, pores may be formed in Al₂O₃ via a reduction reaction at 1,000° C. and at 10⁻¹⁹ P_{O2} atm. The mixture gas of carbon monoxide (CO) and carbon dioxide (CO₂) or hydrogen (H₂) and water (H₂O) has a low partial pressure of oxygen (P_{O2}). A Brunauer-Emmett-Teller (BET) instrument is used to measure the specific surface area and pore size. The measurement of buoyancy and density determines the porosity. A scanning electron microscope (SEM) and energy dispersive spectroscopy (EDS) are used to characterize the sample for microstructure and chemical composition. X-ray diffraction (XRD) is used to measure the crystallographic phase. The CO₂ conversion and methanol (CH₃OH) selectivity/yield are evaluated by gas chromatography (GC) as a function of dielectric packing material porosity.

In another aspect, the present invention is directed to a device for producing valuable materials from carbon dioxide using a dielectric barrier discharge, the device including a reactor body, an internal electrode and an external electrode provided in the reactor, an inlet tube for supplying reactants into the reactor, a power supply for supplying current to the internal electrode and the external electrode to generate plasma and a ground portion of the current connected to the external electrode, wherein the reactor body is packed with the dielectric packing material or the catalyst composite.

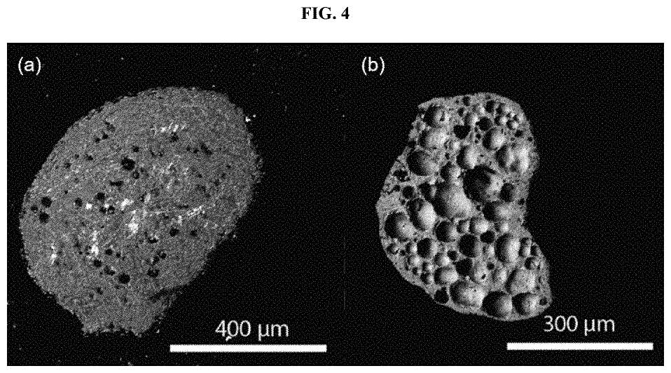

FIG. 3 is a schematic diagram illustrating a plasma device for converting CO₂ into methanol.

The dielectric barrier discharge converts the gas phase into a plasma state, and in the plasma state, reactant molecules are excited, dissociated, or ionized to thereby exhibit high reactivity. The catalytic adsorption behavior of excited molecules is different from that of ground-state molecules. When carbon dioxide, which is a reactant, is adsorbed on the catalyst, the carbon-oxygen binding energy decreases, dissociation can be easily caused by dielectric barrier discharge, and valuable materials can be produced through rapid reaction with hydrogen or water vapor. The present invention enables the catalyst to be activated through excitation, dissociation and ionization processes based on dielectric barrier discharge, thereby rapidly converting carbon dioxide into valuable materials even at a low temperature under atmospheric pressure.

The produced valuable material may be discharged to the outside through an outlet of the reactor, and the discharged valuable material may be analyzed using a gas chromatograph.

In the present invention, any conductive metal may be used as the electrodes, and the internal electrode may be used in any of various forms, which are selected from the group consisting of a general metal wire, a thin metal tube, a metal rod or a metal spring. The external electrode may be a thin metal film as described above and may be in a form that is obtained by coating the outside of the reactor with a metal. In this case, the coating may use a thin metal plate or a metal paste.

In the present invention, any material may be used for the reactor body, so long as it is a tube serving as a dielectric and has dielectric properties.

The power supply may be an AC or pulsed power supply or may be a high-voltage or high-frequency AC power supply.

In the present invention, the device may further include a heater to heat the reactor, and the temperature of the heater may be maintained at 30° C. to 100° C.

In still another aspect, the present invention is directed to a method of preparing valuable materials from carbon dioxide, the method including (a) packing a reactor body with the dielectric packing material or the catalyst composite and supplying a carbon-dioxide-containing gas mixture thereto, (b) heating the supplied gas mixture, and (c) applying a high voltage to the internal electrode and the external electrode provided in the reactor to thereby prepare valuable materials.

In the present invention, the heating in step (b) may be carried out to 100° C. or lower under atmospheric pressure.

In the present invention, the high voltage in step (c) may be 5 to 15 kV.

In the present invention, the carbon-dioxide-containing gas mixture may be a mixture of carbon dioxide and hydrogen or a mixture of carbon dioxide and water vapor, and may further contain nitrogen in addition to the mixture of carbon dioxide and water vapor. The reason therefor is to prevent condensation of water vapor, and the ratio of nitrogen to total gas may be 90 to 95%.

In the present invention, the valuable material may be methanol.

Hereinafter, although preferred embodiments will be described for better understanding of the present invention, it will be obvious to those skilled in the art that these embodiments are provided only for illustration of the present invention, a variety of modifications and alterations are possible without departing from the ideas and scope of the present invention and these modifications and alterations fall within the scope of claims of the present invention.

### EXAMPLE

### Example 1: Production and Characterization of Dielectric Packing Material Having Hollow Therein

In order to optimize the energy efficiency, alumina (Al₂O₃) dielectric packing materials having various shapes, such as spherical, cylindrical and disk shapes, were produced, and the effects thereof were compared.

The diameter, length, and size of each geometry were tested under optimized conditions. Methane conversion was used to identify the optimized geometry and shape.

A Brunauer-Emmett-Teller (BET) instrument was used to measure the specific surface area and pore size. The porosity was determined by measuring the buoyancy and density. A scanning electron microscope (SEM) and energy dispersive spectroscopy (EDS) were used to characterize the sample for microstructure and chemical composition. X-ray diffraction (XRD) was used to measure the crystallographic phase. The CO₂ conversion and methanol (CH₃OH) selectivity/yield were evaluated by gas chromatography (GC) as a function of dielectric packing material porosity using the following Equations (1) to (3) to acquire the conversion, selectivity, and yield:

$$\begin{matrix}[ Equation 1 ] & \\ Conversion Rate ( {CO}_{2} ) = \frac{{CO}_{2} consumed ( mole )}{{CO}_{2} input ( mole )} \times 100 \% & ( 1 ) \\ [ Equation 2 ] & \\ Selectivity ( {CH}_{3} OH ) = \frac{{CH}_{3} OH produced ( mole )}{{CH}_{3} OH converted ( mole )} \times 100 \% & ( 2 ) \\ [ Equation 3 ] & \\ Yield ( {CH}_{3} OH ) = \frac{Selectivity ( {CH}_{3} OH )}{Conversion ( {CO}_{2} )} \times 100 \% & ( 3 )\end{matrix}$$

### Example 2: Production and Characterization of Catalyst Composition Containing Catalyst-Coated Dielectric Packing Material Having Hollow Therein

Catalysts including Au, Pd—ZnO, Pt—In₂O₃, BaTiO₃, and/or Cu-promoted In₂O₃/TiO₂ were used. The sample was characterized using a scanning electron microscope (SEM) to determine the microstructure thereof, energy dispersive spectroscopy (EDS) to determine the chemical composition thereof, and X-ray diffraction (XRD) to determine the crystallographic phase thereof. Tape tests following ASTM standards were used to test the coating adhesion. The CO₂ conversion and methanol yield as a function of catalyst concentration were determined through gas chromatography.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

1. A dielectric packing material for converting carbon dioxide to valuable materials by scattering non-thermal plasma, having a hollow cylindrical structure and multiple grooves on a surface thereof.

2. The dielectric packing material of claim 1, wherein the dielectric packing material is at least one selected from the group consisting of Al₂O₃, ZrO₂, CaO, MgO, zeolite, quartz sand and glass beads.

3. The dielectric packing material of claim 1, wherein the dielectric packing material has a particle size of 180 μm to 300 μm.

4. A catalyst composite for converting carbon dioxide to valuable materials by scattering non-thermal plasma, the catalyst composite having a structure in which a surface of the dielectric packing material of claim 1 is coated with a catalyst.

5. The catalyst composite of claim 4, wherein the catalyst is selected from the group consisting of Cu, Pt, Pd, Au, In₂O₃, ZnO, BaTiO₃ and/or TiO₂.

6. A method of producing a dielectric packing material for converting carbon dioxide to valuable materials by scattering non-thermal plasma of claim 1, the method comprising performing a reduction reaction by sintering a dielectric packing material at a temperature of 1,000° C. to 1,200° C. and an oxygen partial pressure (P_{O2}) of 10⁻⁸ to 10⁻⁶ atm.

7. A method of preparing a catalyst composite for converting carbon dioxide to valuable materials by scattering non-thermal plasma, the method comprising coating a surface of the dielectric packing material of claim 1 with a catalyst.

8. The method of preparing the catalyst composite of claim 7, wherein the coating is dip coating or plasma spray coating.

9. A device of preparing valuable materials from carbon dioxide using a dielectric barrier discharge, the device comprising:
a reactor body;
an internal electrode and an external electrode provided in the reactor;
an inlet tube for supplying reactants into the reactor;
a power supply for supplying current to the internal electrode and the external electrode to generate plasma; and
a ground portion of the current connected to the external electrode,
wherein the reactor body is packed with the dielectric packing material of claim 1 or a catalyst composite comprising said dielectric packing material in which a surface of the dielectric packing material is coated with a catalyst.

10. The device of preparing valuable materials from carbon dioxide of claim 9, further comprising a heater for heating the reactor,
wherein a temperature of the heater is maintained at 30° C. to 100° C.

11. A method of preparing valuable materials from carbon dioxide, the method comprising:
(a) packing a reactor body with the dielectric packing material of claim 1 or a catalyst composite comprising said dielectric packing material in which a surface of the dielectric packing material is coated with a catalyst, and supplying a carbon-dioxide-containing gas mixture thereto;
(b) heating supplied gas mixture; and
(c) preparing valuable materials by applying a high voltage to an internal electrode and an external electrode provided in the reactor.

12. The method of preparing valuable materials from carbon dioxide of claim 11, wherein the heating in step (b) is performed at 100° C. or lower under atmospheric pressure.

13. The method of preparing valuable materials from carbon dioxide of claim 11, wherein the high voltage in step (c) is 5 kV to 15 kV.

14. The method of preparing valuable materials from carbon dioxide of claim 11, wherein the valuable material is methanol.
